(19) United States
(12) Patent Application Publication
(10) Pub. No.: US 2025/0359791 A1
(43) Pub. Date: Nov. 27, 2025

(54) INITIATING COMPUTER ACTIONS BASED ON GAS SENSING

(71) Applicant: Microsoft Technology Licensing, LLC, Redmond, WA (US)

(72) Inventors: Mary P. CZERWINSKI, Kirkland, WA (US); Javier HERNANDEZ RIVERA, Somerville, MA (US); Judith AMORES FERNANDEZ, Somerville, MA (US); Edward Bordeau BROOKS, Chicago, IL (US)

(73) Assignee: Microsoft Technology Licensing, LLC, Redmond, WA (US)

(21) Appl. No.: 18/674,757

(22) Filed: May 24, 2024

(51) Int. Cl. A61B 5/16; A61B 5/00; A61B 5/08; G01N 33/497

(52) U.S. Cl. CPC A61B 5/165; A61B 5/082; A61B 5/6803; A61B 5/6897; A61B 5/7267; A61B 5/7275; A61B 5/7278; A61B 5/746; G01N 33/4975

(57) ABSTRACT

This document relates to causing computers to perform various actions based on gas sensor readings. Gas sensor readings indicating gas levels of various gases can be employed to predict emotional characteristics of a user. For instance, a gas sensor placed near a user's mouth can obtain gas sensor readings indicating levels of nitrogen dioxide, ethyl alcohol, volatile organic compounds, and/or carbon monoxide in the user's breath. Then, gas sensor readings can be used to obtain gas level features, which are input to a trained machine learning model. The trained machine learning model can output a predicted emotional characteristic of the user, such as valence or arousal. Then, a computer can perform an action based on the predicted emotional characteristic. For instance, the action can include adjusting behavior of an automated agent, e.g., to help a distressed user calm down, outputting an alert when the user is in distress, etc.

### BACKGROUND

In some circumstances, it is possible to use video or audio captures to infer the emotional state of a user by correlating facial expressions, gestures, and/or voice characteristics to user emotions. However, the use of cameras and/or microphones to capture a user's environment can implicate privacy concerns. Alternative approaches to sensing user emotions can employ intrusive contact sensors, which tend to be disfavored by many users.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

The description generally relates to computing scenarios involving gas sensing. One example relates to a method or technique that can include obtaining gas sensor readings from a gas sensor, the gas sensor being located in a vicinity of a user when the gas sensor readings are obtained. The method or technique can also include obtaining gas level features, the gas level features being based on the gas sensor readings. The method or technique can also include inputting the gas level features to a trained machine learning model. The method or technique can also include receiving, from the trained machine learning model, a predicted emotional characteristic of the user. The method or technique can also include causing a computer to perform an action based at least on the predicted emotional characteristic of the user.

Another example relates to a method or technique than can include obtaining training data, the training data including gas sensor readings from a gas sensor, the gas sensor being located in a vicinity of a user when the gas sensor readings are obtained and emotional characteristic ratings indicating emotional characteristics of the user when the gas sensor readings are obtained. The method or technique can also include obtaining gas level features, the gas level features being based on the gas sensor readings. The method or technique can also include training a machine learning model to perform gas sensor-based prediction of emotional characteristics based on the gas level features and the emotional characteristic ratings. The method or technique can also include outputting the trained machine learning model.

Another example entails a system that includes a processor and a computer-readable storage medium storing instructions which, when executed by the processor, cause the system to obtain gas sensor readings from a gas sensor, the gas sensor being located in a vicinity of a user when the gas sensor readings are obtained. The instructions can also cause the system to obtain gas level features based on the gas sensor readings. The instructions can also cause the system to input the gas level features to a trained machine learning model. The instructions can also cause the system to receive, from the trained machine learning model, a predicted emotional characteristic of the user. The instructions can also cause the system to perform an action based at least on the predicted emotional characteristic of the user.

The above-listed examples are intended to provide a quick reference to aid the reader and are not intended to define the scope of the concepts described herein.

### BRIEF DESCRIPTION OF THE DRAWINGS

The Detailed Description is described with reference to the accompanying figures. In the figures, the left-most digit(s) of a reference number identifies the figure in which the reference number first appears. The use of similar reference numbers in different instances in the description and the figures may indicate similar or identical items.

FIG. 1 illustrates an example system, consistent with some implementations of the present concepts.

FIG. 2 illustrates an expanded view of a gas sensor array on a wearable device, consistent with some implementations of the present concepts.

FIG. 3 illustrates an example scenario in which the present concepts can be employed.

FIG. 4 illustrates an example neural network that can be trained to predict emotional characteristics based on gas level features, consistent with some implementations of the present concepts.

FIG. 5 illustrates an example random forest that can be trained to predict emotional characteristics based on gas level features, consistent with some implementations of the present concepts.

FIG. 6 illustrates an example method or technique for causing a computer to perform an action based on a predicted emotional characteristic of a user, consistent with some implementations of the disclosed techniques.

FIG. 7 illustrates an example method or technique for training a machine learning model to perform gas sensor-based prediction of emotional characteristics based on gas level features, consistent with some implementations of the disclosed techniques.

FIGS. 8A, 8B, and 8C illustrate experimental results obtained using some implementations of the present concepts.

FIGS. 9A and 9B illustrate example user experiences, consistent with some implementations of the disclosed techniques.

### DETAILED DESCRIPTION

As noted previously, one way to determine the emotional state of a user is to employ a camera to monitor a user's facial expression and/or gestures. Another approach involves using microphones to monitor voice activity. However, recording a user's environment with a camera or microphone can result in inadvertent leaking of private information. Other technologies aim to use contact sensors to measure signals such as users' heart rate, blood pressure, or neurological signals. However, contact sensors are relatively intrusive and disfavored by many users.

The disclosed implementations provide a private, non-contact approach for sensing the emotional states of users via gas sensing. For instance, as described more below, one or more gas sensors can detect gases such as nitrogen dioxide, ethyl alcohol, volatile organic compounds, and/or carbon monoxide. These gases can be detected using gas sensors that can be provided in a wearable device that senses gas levels in the user's breath. Some implementations can also provide one or more other gas sensors located further from the user, e.g., to detect the ambient levels of gases in the user's environment.

The disclosed implementations can train a machine learning model to predict emotional characteristics of users from the levels of various gases detected by one or more gas sensors. At inference time, the trained machine learning model can be employed to cause computers to perform a wide range of actions based on predicted emotional characteristics of users. For instance, the trained machine learning model can provide a basis for applications ranging from emotion-aware chatbots to automatically alerting clinicians when a coma patient is in discomfort.

### Machine Learning Overview

There are various types of machine learning frameworks that can be trained to perform a given task, such as performing gas sensor-based prediction of emotional characteristics of users. Support vector machines, decision trees, random forests, and neural networks are just a few examples of machine learning frameworks that have been used in a wide variety of applications, such as image processing and natural language processing.

A support vector machine is a model that can be employed for classification or regression purposes. A support vector machine maps data items to a feature space, where hyperplanes are employed to separate the data into different regions. Each region can correspond to a different classification. Support vector machines can be trained using supervised learning to distinguish between data items having labels representing different classifications.

A decision tree is a tree-based model that represents decision rules using nodes connected by edges. Decision trees can be employed for classification or regression and can be trained using supervised learning techniques. Multiple decision trees can be employed in a random forest, which significantly improve the accuracy of the resulting model relative to a single decision tree. In a random forest, the individual outputs of the decision trees are collectively employed to determine a final output of the random forest. For instance, in regression problems, the output of each individual decision tree can be averaged to obtain a final result. For classification problems, a majority vote technique can be employed, where the classification selected by the random forest is the classification selected by the most decision trees.

A neural network is another type of machine learning model that can be employed for classification or regression tasks. In a neural network, nodes are connected to one another via one or more edges. A neural network can include an input layer, an output layer, and one or more intermediate layers. Individual nodes can process their respective inputs according to a predefined function, and provide an output to a subsequent layer, or, in some cases, a previous layer. The inputs to a given node can be multiplied by a corresponding weight value for an edge between the input and the node. In addition, nodes can have individual bias values that are also used to produce outputs.

Various training procedures can be applied to learn the edge weights and/or bias values of a neural network. The term “internal parameters” is used herein to refer to learnable values such as edge weights and bias values that can be learned by training a machine learning model, such as a neural network. The term “hyperparameters” is used herein to refer to characteristics of model training, such as learning rate, batch size, number of training epochs, number of hidden layers, activation functions, etc.

A neural network structure can have different layers that perform different specific functions. For example, one or more layers of nodes can collectively perform a specific operation, such as pooling, encoding, decoding, alignment, prediction, or convolution operations. For the purposes of this document, the term “layer” refers to a group of nodes that share inputs and outputs, e.g., to or from external sources or other layers in the network. The term “operation” refers to a function that can be performed by one or more layers of nodes. The term “model structure” refers to an overall architecture of a layered model, including the number of layers, the connectivity of the layers, and the type of operations performed by individual layers. The term “neural network structure” refers to the model structure of a neural network. The term “trained model” and/or “tuned model” refers to a model structure together with internal parameters for the model structure that have been trained or tuned, e.g., individualized tuning to one or more particular users. Note that two trained models can share the same model structure and yet have different values for the internal parameters, e.g., if the two models are trained on different training data or if there are underlying stochastic processes in the training process.

### Definitions

A “gas sensor” is a sensor adapted to detect concentration levels of one or more gases in an environment. For instance, gas sensors can detect nitrogen dioxide levels, ethyl alcohol levels, volatile organic compound levels, carbon monoxide levels, or levels of any other gas in an environment. A “feature” is a value that can be processed by a machine learning model to predict a value. A “gas level feature” is a feature that corresponds to the level of one or more gases as measured by a gas sensor. For instance, a gas level feature could convey the current level of a particular gas in an environment, the change in the level of a particular gas in the environment over time, or a statistical measure (mean, standard deviation, etc.) determined by readings from a gas sensor. As discussed more below, gas level features can be useful for predicting various emotional characteristics of a user, because the level of certain gases in a user's breath and/or emitted from other parts of their body can correlate to specific emotional characteristics. A gas sensor is within a “vicinity” of a user when the gas sensor is close enough to the user to be employed to predict emotional characteristics of the user, depending on the sensitivity of the gas sensor(s) and/or precision/accuracy of the model. In the experiments described below, a gas sensor measuring a user's breath was placed approximately 5 centimeters away from their mouth, whereas a gas sensor measuring ambient gas levels was placed on a desk approximately 1 meter from the user.

A “respiration feature” is a feature that conveys characteristics of a user's breath. For instance, a respiration feature can convey the duration or intensity (e.g., volume of inhaled or exhaled air) of one or more breaths by a user. Respiration features can also include statistical values calculated over multiple breaths by a user. Respiration features can also be useful for predicting emotional characteristics of users, e.g., users may breathe more quickly or heavily when under stress than when they are relaxed.

A “bio-signal feature” is a feature that conveys some biological information about the user. For instance, a bio-signal feature could be obtained from an electroencephalogram (EEG) signal measuring neurological activity of a user, from an eye tracking sensor measuring a pupil diameter measurement or eye movement of a user, from a photoplethysmography (PPG) sensor measuring a user's heart rate, heart rate variability (HRV), blood oxygenation, and/or blood pressure, etc. Body temperature, facial expressions, gestures, movement dynamics, etc. can also be used to determine bio-signal features. Note that the gases emitted from a user's breath or from other parts of their body can also be considered a type of bio-signal feature, but gases are generally discussed separately from other types of bio-signal features herein.

A “context feature” is a feature that characterizes a context of a user. For instance, a context feature could convey an application that is running when a user is involved with a computer, input by the user to the computer using a mouse, keyboard, touchscreen, voice, or any other information relating to user interaction with a computer or other system. A context feature could also identify the current time of day, season or month of the year, the location of the user, their age, gender, health status, etc. A context feature could also identify social media contacts of a user, their profession, educational level, or any other information about a user that can be employed to predict their emotional characteristics and/or cause a computer to perform an action for the user.

An “emotional characteristic” of a user can be an emotion itself, such as happiness, anger, sadness, surprise, distress, boredom, calmness, relaxation, excitement, disgust, amusement, etc. An emotional characteristic can also be an emotional dimension, such as valence or arousal. Some techniques for characterizing emotions map emotions to valence and arousal dimensions. Thus, for example, being depressed can be an emotion with negative valence and low arousal, being angry can be an emotion with negative valence and high arousal, being excited can be an emotion with positive valence and high arousal, and being relaxed can be an emotion with positive valence and low arousal. As described more below, a machine learning model can be trained to predict emotional dimensions such as valence and arousal, or to directly predict emotions without regard to emotional dimensions.

The term “emotional content” refers to content that tends to elicit a specific emotional response from users. For instance, videos of surgery tend to elicit a disgusted response from users, funny animal videos tend to elicit an amused response from users. Audio clips or passages of reading material can also be employed as emotional content.

An “application” is a computing program that runs locally or remotely from a user. An application can be a virtual reality application that immerses the user entirely or almost entirely in a virtual environment. An application can also be an augmented reality application that presents virtual content in a real-world setting. Other examples of applications include productivity applications (e.g., word processing, spreadsheets), video games, digital assistants or chatbots, teleconferencing applications, email clients, web browsers, operating systems, Internet of Things (IoT) applications, etc.

The term “model” is used generally herein to refer to a range of processing techniques, and includes models trained using machine learning as well as hand-coded (e.g., heuristic-based) models. For instance, as noted above, a machine-learning model could be a neural network, a support vector machine, a decision tree, a random forest, etc. Models can be employed for various purposes as described below, such as gesture classification.

### Example System

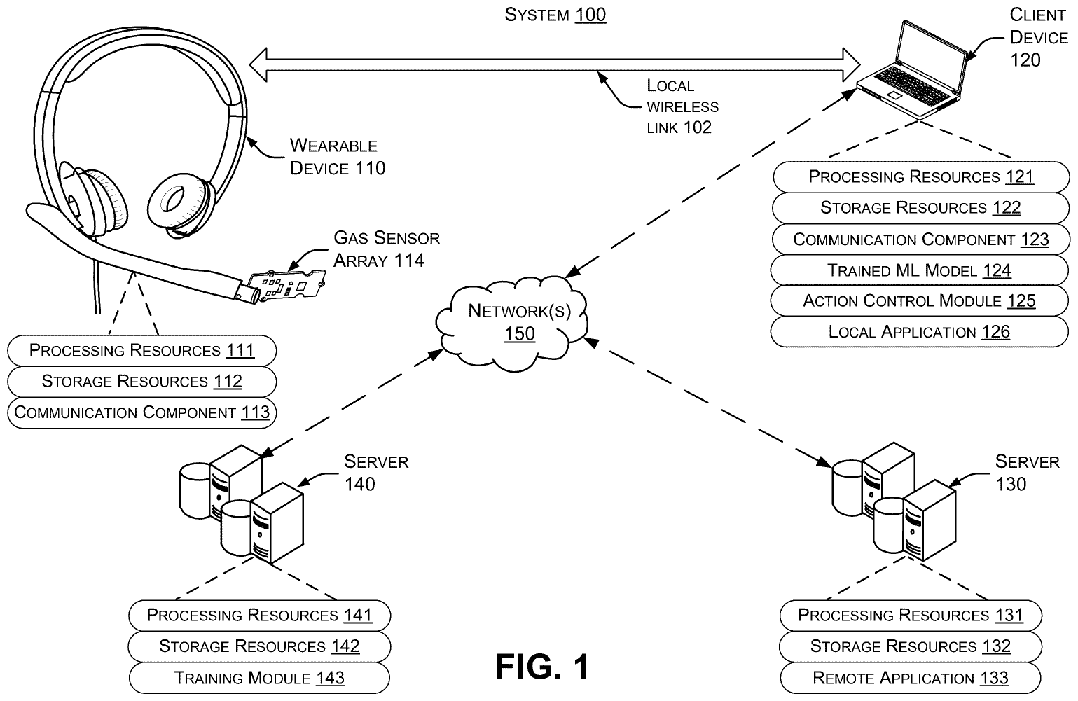

The present implementations can be performed in various scenarios on various devices. FIG. 1 shows an example system 100 in which the present implementations can be employed, as discussed below.

As shown in FIG. 1, system 100 includes a wearable device 110, a client device 120, a server 130, and a server 140. Wearable device 110 and client device 120 are connected by a local wireless link 102. Client device 120, server 130, and server 140 are connected by one or more network(s) 150. Note that the client device can be embodied as a mobile device such as a smart phone or tablet, or as a laptop, desktop, blade or rack server, etc. Likewise, the servers can be implemented using various types of computing devices. In some cases, any of the devices shown in FIG. 1, but particularly the servers, can be implemented in data centers, server farms, etc.

Wearable device 110 can have processing resources 111 and storage resources 112, client device 120 can have processing resources 121 and storage resources 122, server 130 can have processing resources 131 and storage resources 132, and server 140 can have processing resources 141 and storage resources 142. Each of these devices may also have various modules that function using the processing and storage resources to perform the techniques discussed herein. The processing resources can include central processing units (“CPUs”), graphics processing units (“GPUs”), neural processing units (“NPUs”), etc. The storage resources can include both persistent storage resources, such as magnetic or solid-state drives, and volatile storage, such as one or more random-access memory devices. In some cases, the modules are provided as executable instructions that are stored on persistent storage devices, loaded into the random-access memory devices, and read from the random-access memory by the processing resources for execution.

The wearable device 110 can include a communication component 113. The communication component can obtain gas sensor readings from gas sensor array 114 and send the gas sensor readings to client device 120. The client device 120 can receive the gas sensor readings with communication component 123, process the gas sensor readings to obtain gas level features, and input the gas level features into trained machine learning model 124. In some implementations, the gas sensor readings can include a voltage that changes as the sensor resistance fluctuates in response to changing gas concentrations. Then, the measured voltages can be used to calculate the concentrations of various gases, which in turn can be used to derive the gas level features. The trained machine learning model can predict one or more emotional characteristics of a user based on the gas level features. The predicted emotional characteristics can be used by action control module 125 to control a local application 126 and/or a remote application 133 on server 130. For instance, the action control module can employ one or more rules that map predicted emotional characteristics to actions to be performed, or else can employ machine learning to determine which actions to perform based on predicted emotional characteristics. In other implementations, the action control module is provided in an operating system that communicates the predicted emotional characteristics to the local application or a remote application that takes one or more of the actions described herein based on the predicted emotional characteristics. Also, note that in some cases the wearable device 110 can perform local inference using the trained machine learning model (e.g., on an NPU) and send predicted emotional characteristics instead of gas sensor readings to the client device. In other cases, the action control module and/or local application can also be implemented on the wearable device.

Server 140 can include a training module 143 that trains a machine learning model. The trained machine learning model can be distributed to client device 120 and/or wearable device 110 for predicting emotional characteristics. As described more below, the machine learning model can be trained by obtaining gas sensor readings from a gas sensor and emotional characteristic ratings from users when the gas sensor readings are obtained.

As discussed more below, system 100 is merely an example and additional devices and/or sensors can be employed in systems consistent with the disclosed techniques. For instance, additional gas sensor and/or other types of sensors can be provided, as well as cameras, microphones, etc. Other sensors, cameras, and/or microphones can communicate with wearable device 110 and/or client device 120 using wired or wireless technologies to provide additional features usable by trained machine learning model 124 to predict emotional characteristics of users.

### Example Gas Sensor

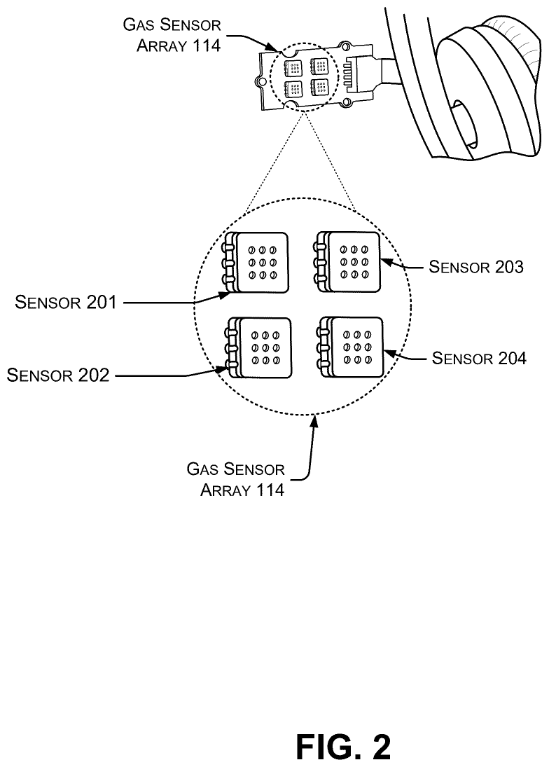

FIG. 2 shows an expanded view of wearable device 110 with additional details on the gas sensor array 114. The gas sensor array is shown with sensor 201, sensor 202, sensor 203, and sensor 204. For example, sensor 201 can be configured to detect carbon monoxide (CO) levels, sensor 202 can be configured to detect nitrogen dioxide (NO2) levels, sensor 203 can be configured to detect ethyl alcohol levels, and sensor 204 can be configured to detect levels of volatile organic compounds.

In some implementations, each sensor is a metal-oxide (MOX) gas sensor or chemiresistor. Generally speaking, MOX chemiresistors are cost-effective and compact. MOX chemiresistors detect changes in electrical resistance when a metal oxide surface absorbs oxygen in the presence of gases, and MOX chemiresistors are practical for integration into interactive computing systems. By adjusting the surface coating, thickness, or shape, MOX sensors can be fine-tuned to better detect certain gases such as the four gases mentioned above. In further implementations, a system-on-a-chip can be employed where individual MOX sensors are integrated into a single circuit with on-chip memory and processing circuitry.

### Example Use Scenario

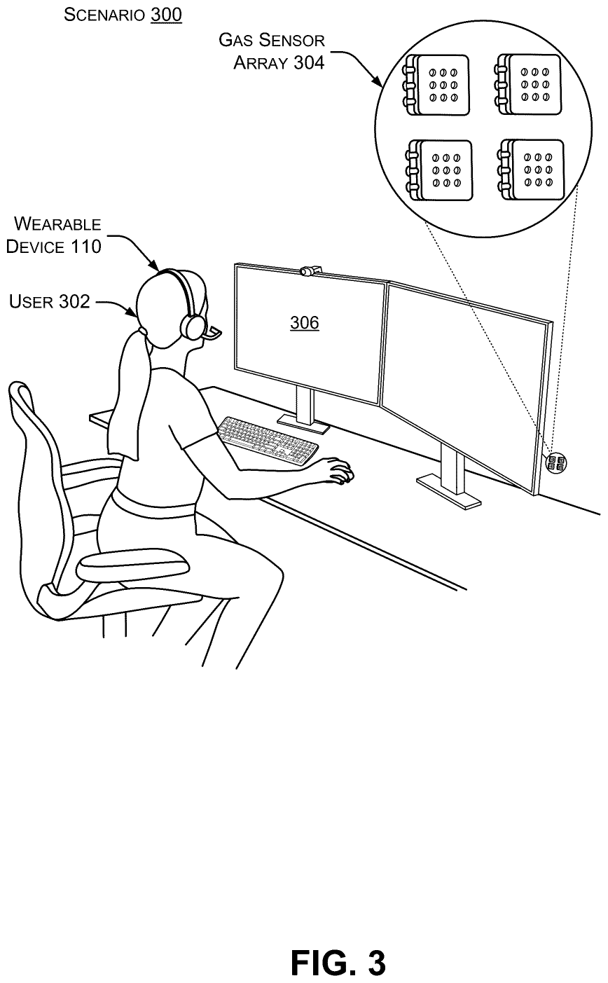

FIG. 3 shows an example scenario 300. Here, a user 302 is seated with wearable device 110. A gas sensor array 304 is also provided on monitors 306. The wearable device may sense local gas levels from the user's breath, while the gas sensor array 304 may sense ambient gas levels within the room. The gas sensor array 304 may communicate the ambient gas levels to the wearable device 110 or to a client device (not shown in FIG. 3) connected to one of the monitors.

The use of a separate gas sensor array 304 in conjunction with gas sensor array 114 on the wearable device 110 can be useful for several reasons, discussed in more detail below. Generally speaking, the gas sensor array 114 on the wearable device can quickly detect changes to the gas composition of a user's breath. On the other hand, gas sensor array 304 can detect ambient gas levels that can change more slowly over time, as a result of the user's breath, other bodily emissions of the user, as well as the breath and/or other bodily emissions of other users that may be in the room.

### First Example Machine Learning Model

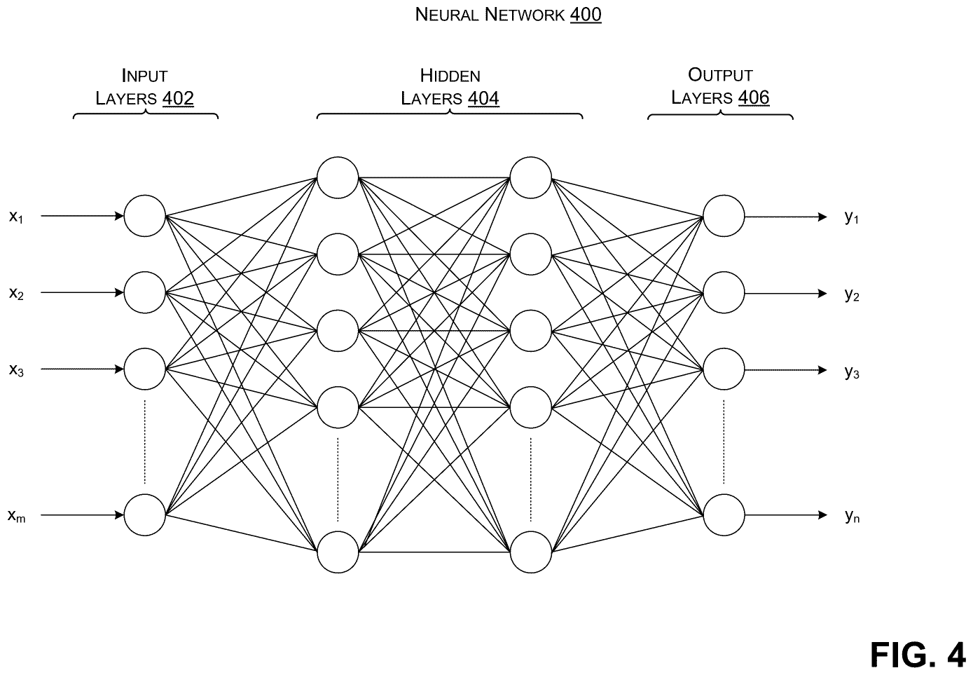

FIG. 4 shows a deep neural network 400 with input layers 402, hidden layers 404, and output layers 406. The input layers can receive features x₁ through xₘ. For instance, the features can include gas level features and/or respiration features obtained from a gas sensor. The features can also include other types of features, such as biosensor features and/or context features.

The input layers can feed into the hidden layers 404. The hidden layers feed into the output layers 406. The output layers can output values y₁ through yₘ. For instance, the output values can characterize emotional characteristics of a user. In some cases, the output values are calculated using a regression approach, and in other cases using a classification approach.

In a regression approach, the output values can characterize emotional characteristics of a user using a numerical value. For instance, one output layer could generate a value indicating a predicted valence rating of a user based on a set of input features, and another output layer could generate a value indicating a predicted arousal rating of the user based on the set of input features. During training, the internal parameters (e.g., weights and/or bias values) of the deep neural network 400 can be adjusted (e.g., using backpropagation) based on a difference between the predicted ratings and actual valence and arousal ratings provided in training data.

In a classification approach, the output values can include probability distributions over emotional characteristics. For instance, one output layer could output a binary probability distribution that a user is happy, another output layer could output a binary probability distribution that the user is angry, etc. During training, the internal parameters (e.g., weights and/or bias values) of the deep neural network 400 can be adjusted (e.g., using backpropagation) based on a difference between emotional classifications provided by users (e.g., happy, angry, etc.) and the emotional characteristics predicted by the output layers.

### Second Example Machine Learning Model

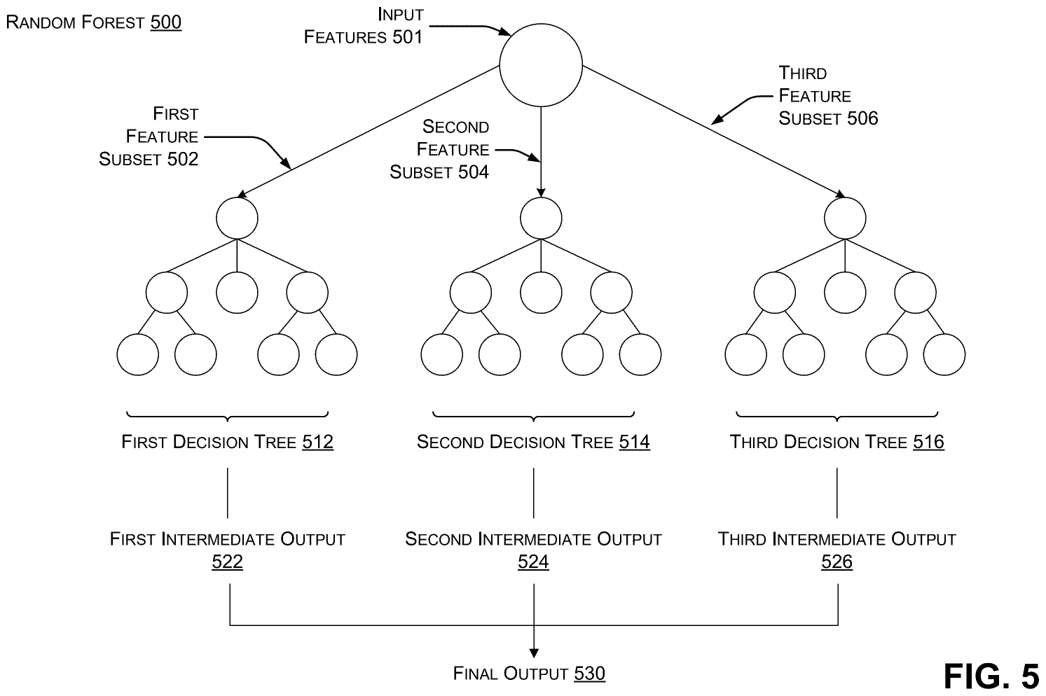

FIG. 5 shows a random forest 500. Input features 501 are distributed as first feature subset 502 to a first decision tree 512, second feature subset 504 to a second decision tree 514, and third feature subset 506 to a third decision tree 516. For instance, the input features can include gas level features and/or respiration features obtained from a gas sensor. The features can also include other types of features, such as biosensor features and/or context features. The feature subsets for each decision tree can be selected using a random approach, where each decision tree gets a different subset of input features.

The first decision tree generates a first intermediate output 522, the second decision tree generates a second intermediate output 524, and the third decision tree generates a third intermediate output 526. The intermediate outputs are combined to generate a final output 530. For instance, in a regression approach, each decision tree predicts a numerical value representing the magnitude of an emotional characteristic (e.g., valence or arousal) of a user based on the subset of input features assigned to that decision tree. The final output can be calculated (e.g., by averaging) the magnitudes output by the respective decision trees. In a classification approach, each decision tree predicts an emotional characteristic (e.g., angry, sad, etc.) of a user based on the subset of input features assigned to that decision tree. The final output can be determined by majority vote of the respective decision trees. Each individual decision tree is trained to determine splitting criteria for its respective input features, where the splitting criteria are used to determine paths through the decision tree to arrive at the intermediate outputs, provided by the leaf nodes of the decision trees.

### Example Inference Time Method

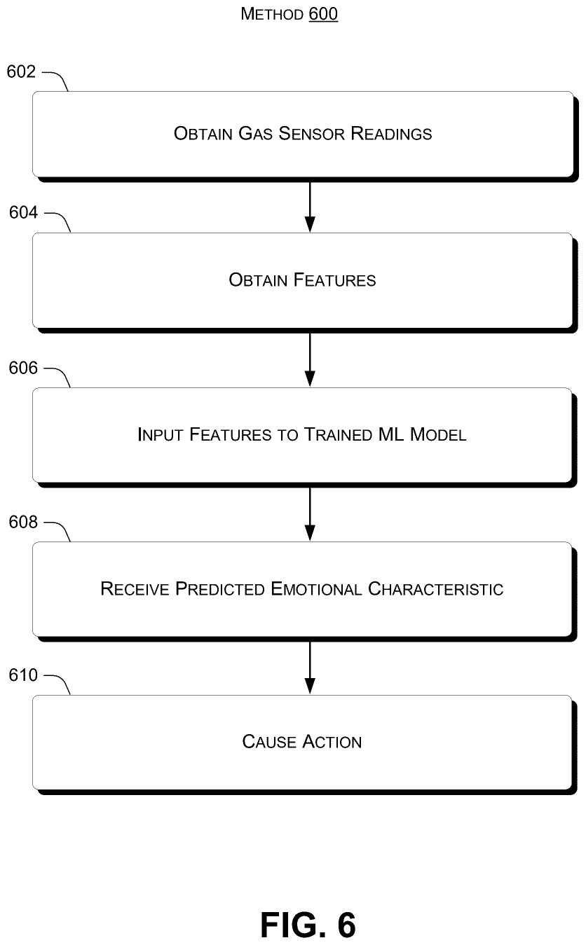

FIG. 6 illustrates an example method 600, consistent with some implementations of the present concepts. Method 600 can be implemented on many different types of devices, e.g., by one or more wearable devices, by one or more cloud servers, by one or more client devices such as laptops, tablets, or smartphones, or by combinations of one or more wearable devices, servers, client devices, etc.

Method 600 begins at block 602, where gas sensor readings are obtained. For instance, as described above, the gas sensor readings can be obtained from a gas sensor array located near a user's mouth to sense the components of the user's breath. As also noted, gas sensor readings can also be obtained from a gas sensor located further from a user, to sense ambient gas levels in a room where the user is located.

Method 600 continues at block 604, where features are obtained. For instance, the features can include gas level features that characterize levels of individual gases measured by the gas sensor(s), changes in gas levels over time, and/or statistical values computed from the gas levels. The features can also include respiration features that characterize breathing by the user. The features can also include context features relating to the user and/or bio-signal features obtained from other sensors.

Method 600 continues at block 606, where the features are input to a trained machine learning model. As described above, deep neural networks and random forests are but two examples of machine learning models that can be employed.

Method 600 continues at block 608, where a predicted emotional characteristic is received from the machine learning model. For instance, the predicted emotional characteristic can convey components of emotions, such as valence and/or arousal. In other implementations, the predicted emotional characteristic can convey a specific predicted emotion such as distress, happiness, sadness, anger, surprise, etc. The predicted emotional characteristic can be conveyed using a regression approach, e.g., numerical values indicating predicted valence and/or arousal ratings of the user. The predicted emotional characteristic can also be conveyed using a classification approach, a binary value indicating whether the user is predicted to be happy, sad, angry, etc.

Method 600 continues at block 610, where at least one computer action is caused based on the predicted emotional characteristic. For instance, the action can include adjusting the behavior of an automated agent (e.g., chatbot or generative model) assisting the user, triggering an alert regarding the user, controlling an environment where the user is located, displaying content to the user, etc. In some cases, the action is determined by an application that receives the predicted emotional characteristic from an operating system via an application programming interface, and then the application determines which action to perform based on the predicted emotional characteristic.

In some cases, method 600 can be performed entirely by a wearable device or a client device. In other cases, parts of method 600 are performed on different computing devices. For instance, in some cases, a wearable device can perform blocks 602, 604, 606, and 608, and then send the predicted emotional characteristic to a client device that performs block 610.

### Example Training Method

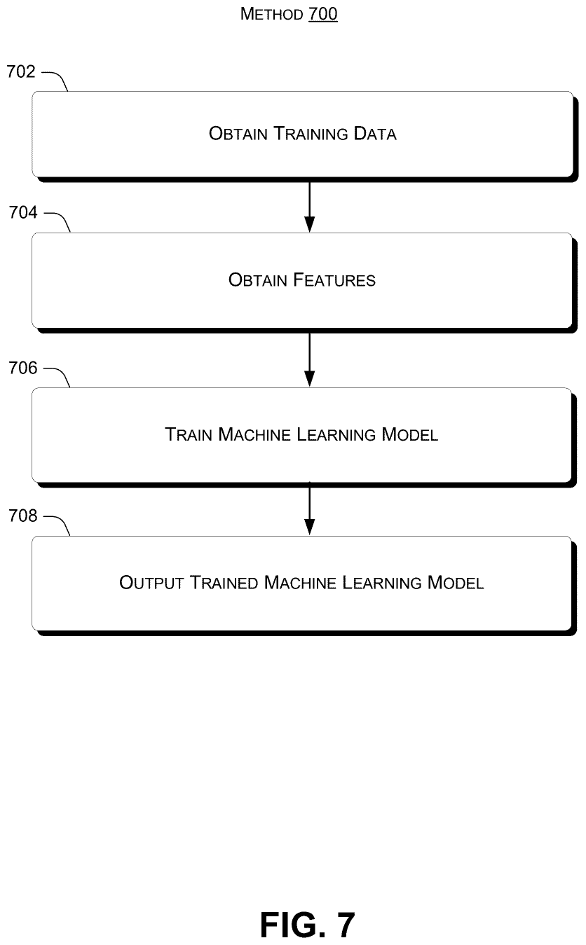

FIG. 7 illustrates an example method 700, consistent with some implementations of the present concepts. Method 700 can be implemented on many different types of devices, e.g., by one or more wearable devices, by one or more cloud servers, by one or more client devices such as laptops, tablets, or smartphones, or by combinations of one or more wearable devices, servers, client devices, etc.

Method 700 begins at block 702, where training data is obtained. For instance, the training data can include gas sensor readings from a gas sensor in the vicinity of a user. The training data can also include emotional characteristic ratings obtained from the user. For instance, the user can rate their valence and/or arousal on a numerical scale while viewing emotional content as the gas sensor readings are obtained. In other cases, the emotional characteristic ratings can be binary values indicating whether the user feels a specific emotion, e.g., happiness, sadness, disgust, etc.

Method 700 continues at block 704, where features are obtained. The features can include gas level features that characterize levels of individual gases measured by the gas sensor(s), changes in gas levels, or statistical values computed from the gas levels. The features can also include respiration features that characterize breathing by the user. The features can also include context features relating to the user and/or bio-signal features obtained from other sensors.

Method 700 continues at block 706, where a machine learning model is trained. For example, a neural network or random forest can be trained using a regression approach to predict numerical values conveying emotional characteristics of users. In other cases, a neural network or random forest can be trained using a classification approach to classify emotional characteristics of uses.

Method 700 continues at block 708, where the trained machine learning model is output. For instance, the trained machine learning model can be stored on persistent storage for subsequent local inference processing, distributed over a network to one or more other devices for inference processing, etc.

In some cases, method 700 can be performed entirely by a single computing device, such as server 140. In other cases, parts of method 700 are performed on different computing devices.

### Specific Implementations

The following discussion describes some specific implementations of the disclosed concepts using a regression approach. Also described is the Affective Air Quality (AAQ) dataset, a dataset including volatile odor compound and gas sensor data that was collected and employed for non-contact emotion detection. The AAQ dataset includes 4-channel gas sensor data obtained from 23 participants at two distances from the body. The sensors were positioned on a wearable device to detect gas levels in the users' breath, and on a desktop to collect ambient gas levels. The gas sensor readings were collected with emotional characteristic ratings elicited by targeted movie clips. The AAQ dataset was employed to analyze the correlation between personal chemical emissions and varied emotional responses. Participants viewed emotional content (e.g., video clips) designed to elicit strong emotional responses. Participants rated their own valence, arousal, and familiarity on a scale of 1 through 9, while gas sensor readings were obtained. Familiarity ratings can be useful to discern when users may have relatively muted emotional reactions to content because they are already familiar with the content.

Using changes in breath and body emission composition to detect emotions is an unobtrusive approach that mitigates privacy and comfort concerns associated with methods that rely on audio-visual input or direct physical contact. The AAQ dataset captures real-time volatile odor compound (VOC) and gas sensor data (personal chemical emissions) while participants experience different emotional responses elicited by movie clips in a controlled setting. Sensor data was obtained by two custom-made devices (a wearable and a standalone), that use off-the-shelf metal oxide gas sensors.

1) Hardware: Chemical signals were captured using the Grove Multichannel Gas Sensor (v2), which includes four MOX gas sensors each optimized for different gases but generally non-selective towards oxidizing gases. These sensors are responsive to nitrogen dioxide (Winsen GM102B), ethanol (Winsen GM-302B), volatile organic compounds (Winsen GM-502B), and carbon monoxide (Winsen GM-702B), yet can detect a similar range of gases. With ethanol, 2-propranol and 1-propanol identified as stress-sensitive compounds, MOX sensors can be employed for stress detection.

To ensure thorough coverage of the testing area, gas sensors were placed in two locations: directly in front of the participant's nose and mouth, and on the study desk about 0.75 m from the participant. The sensor near the nose and mouth was attached to a microphone goose-neck on headphones. To prepare the gas sensor data for feature extraction, several pre-processing steps were implemented. A fourth order band-pass Butterworth filter with a 30 Hz cutoff was employed to attenuate high and low-frequency noise, followed by an Exponential Weighted Moving Average (EWMA) with α=0.08. Finally, min-max normalization was applied to standardize the data range across channels, per video clip.

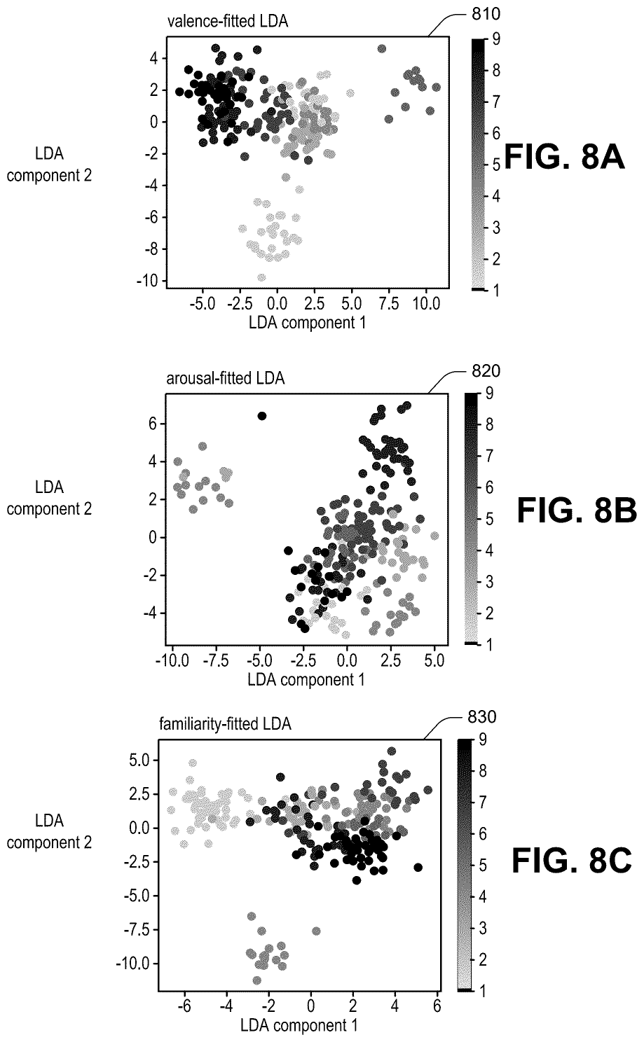

Note that while the experiments discussed herein did not track participants' respiration, the averaged signal across the four headset gas sensors could be used to estimate respiration cycles. For instance, a moving average filter could average the signal over a set number of samples to smooth the signal and then identify the peaks related to inhalation and exhalation. The time interval between successive peaks is one respiration cycle, which can be used to determine the respiration rate or number of breaths per minute. Alternatively, gas sensor signals during a single exhalation or full respiration cycle could also be used. The gas sensor signals measured from both the headset and desktop AQ monitors were measured during emotion-eliciting clips. Features extracted included statistical measures (mean, standard deviation, area under curve, skewness, kurtosis), conductance changes, relative abundance (separated by device), mean & standard deviation of each discrete wavelet transform (DWT) coefficient, and Renyi entropy for each gas sensor. Additionally, the sensor response metrics (peak-to-peak time, trough-to-peak signal, and minimum and maximum slope) for both the unnormalized and normalized gas sensor signals was also measured, generating in total a 248D feature vector. FIG. 8A shows a Linear Discriminant Analysis (LDA) plot 810 illustrating separability of arousal scores, FIG. 8B shows an LDA plot 820 illustrating separability of valence scores, and FIG. 8C shows an LDA plot 830 illustrating separability of familiarity scores.

Several different machine learning models were evaluated for emotion detection using the features described above. The models included a support vector machine (SVM) with a radial basis function kernel, a random forest (RF) with 100 decision trees, a gradient-boosted decision tree (XGBoost), and a long short-term neural network (LSTM). Each model was evaluated using a 5-fold cross-validation approach. The problem was treated as a regression task, for which each model was separately trained to predict the valence or arousal from a sequence of features. The LSTM model, designed to address the data's temporal aspect, included two 100-unit layers, processed the last 30 seconds of sensor signals, and featured Dropout layers to reduce overfitting. The network used the GlorotUniform initializer and LeakyReLU activation, concluding with a Dense layer for valence or arousal predictions. Training involved the Adam optimizer and mean squared error loss, with early stopping to curtail overfitting after 50 epochs or 10 epochs without loss improvement. For the cross-validation, all samples were randomly divided into five folds, and in each validation, four folds are used for training and the remaining fold for testing.

The Mean Absolute Error (MAE) and Root Mean Squared Error (RMSE) were determined as metrics, given their direct interpretability in the context of numerical valence and arousal prediction. MAE provides a clear measure of average prediction accuracy in the same units as the target variable, making it particularly relevant for applications in affective computing where precision in emotional state prediction is useful. The RMSE complements the MAE metric by providing a relatively high weight to large errors, making it useful to evaluate a model when large errors are particularly undesirable. The final metric chosen was R², which represents the proportion of the variance in the dependent variable that is predictable from the independent variable.

Table 1 below:

| | Valence | | | Arousal | | |
|---|---|---|---|---|---|---|
| Model | MAE | RMSE | R² | MAE | RMSE | R² |
| SVM | 2.30 ± 0.17 | 2.64 ± 0.19 | 0.03 ± 0.05 | 1.85 ± 0.27 | 2.28 ± 0.28 | −0.03 ± 0.07 |
| RF | 1.93 ± 0.15 | 2.32 ± 0.19 | 0.25 ± 0.08 | 1.80 ± 0.31 | 2.17 ± 0.30 | 0.07 ± 0.04 |
| XGBOOST | 2.09 ± 0.23 | 2.57 ± 0.23 | 0.06 ± 0.19 | 1.82 ± 0.28 | 2.22 ± 0.28 | 0.02 ± 0.16 |
| LSTM | 2.43 ± 0.23 | 2.72 ± 0.18 | −0.05 ± 0.06 | 1.90 ± 0.22 | 2.33 ± 0.28 | −0.07 ± 0.13 |

shows the prediction results of the preliminary models trained. From the table, the model predictions were typically within two units of the actual valence arousal levels on a scale from 1 to 9. The models' RMSE averaging around 2-3 across folds further supports their efficacy by indicating low dispersion of errors. Here, the MAE and RMSE's coarse accuracy indicates that gas sensors can be used effectively in affective computing applications.

### Example User Experience

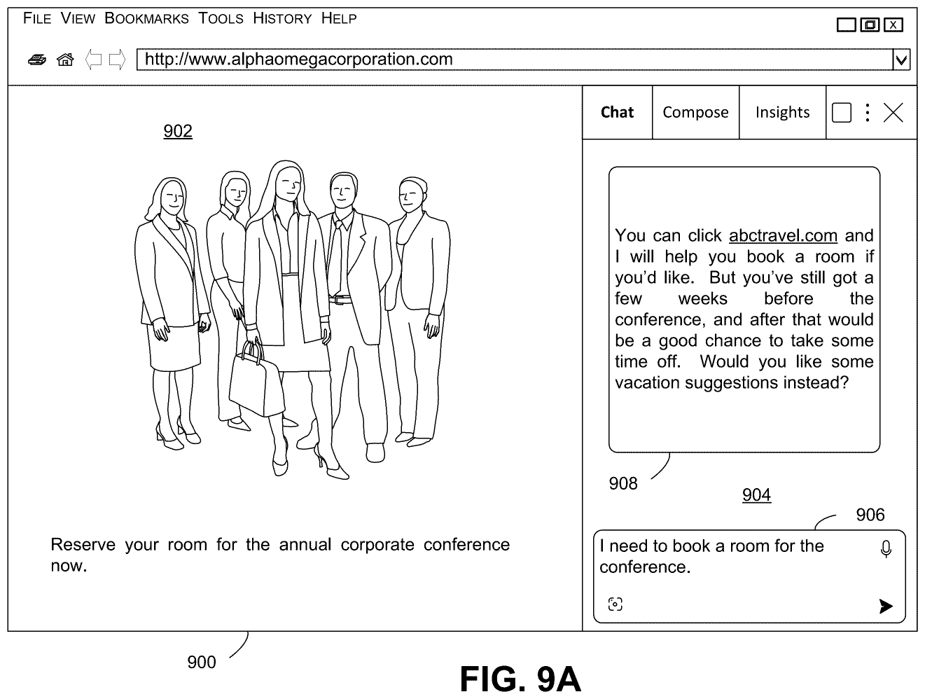

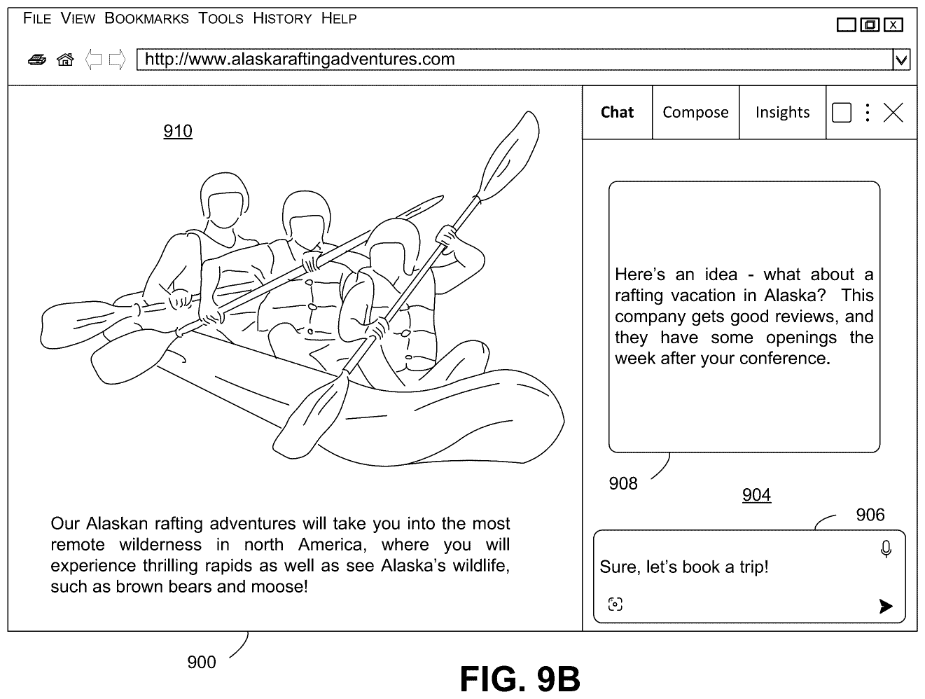

Emotional characteristics predicted using the disclosed techniques can be employed for a wide range of applications. FIG. 9A shows an example user experience, where a browser window 900 includes a first web page 902 and a digital assistant interface 904. The user enters a current natural language query “I need to book a room for the conference” into prompt area 906. In this example, assume that the user is predicted to be experiencing stress based on gas sensor readings, potentially along with signals from one or more other sensors. Thus, a generative language model generates a response in response area 908. Here, the generative language model gives the user the option to continue with booking the room. However, because the user is determined to be in a stressed emotional state, the generative language model also suggests an alternative for the user to book a vacation instead.

As shown in FIG. 9B, if the user accepts the suggestion by stating “Sure, let's book a trip,” the generative language model can take the user to a second webpage 910. The second webpage can allow the user to book a trip for a rafting vacation. In this case, the response of the generative language model is conditioned on the predicted emotional characteristic of the user. For instance, this could be implemented by prompting the generative language model to respond to the user query in an emotionally-appropriate manner, given that the user is predicted to be experiencing significant stress.

### Further Implementations

The specific implementations described above are just a few of the ways that the disclosed concepts can be employed. For instance, the machine learning model structures shown above are merely exemplary, and other model structures can be employed. As one example, consider an LSTM network with a sliding window that captures multiple (e.g., 2-3) breathing cycles. In further implementations, a bidirectional LSTM could be employed so that features occurring after a change in a user's emotions could be employed to detect the change.

Furthermore, additional features can be used for predicting emotional characteristics. The LDA plots shown above indicate improved separation when combining data from both headset and desktop sensors and including trough/peak features. As another example, additional types of features can also be employed relating to breathing cycles, e.g., over one or more adsorption/absorption cycles. These features can include peak-to-peak features, the ratio of one sensor's signal to total response of all sensors, max conductance change from baseline, Renyi entropy, autocorrelation, auto-mutual information, wavelet entropy, the Generalized Lorentzian, etc. Other features can include Lyapunov exponent, Discrete Wavelet Transform (DWT) coefficients, and features based on the transient response of MOX gas sensor signals.

In addition, as noted previously, gas sensors can be placed further away from a user to sense ambient gas levels in an environment. This can be useful for several reasons. First, human bodies emit chemical signatures from other parts of the body besides the mouth. Second, gases can accumulate over time and thus ambient gas levels accumulated from emissions by the user's mouth and other parts of their body can be more accurately measured further away from the user's mouth. In addition, in cases where a group of multiple users occupies a space such as a meeting room, then the gas levels within that space can convey information about emotional characteristics of the group as a whole. In some cases, gas sensors are provided in heating, ventilation, and air conditioning (“HVAC”) equipment to monitor gases for groups of users in a building.

In addition, there are other types of privacy-preserving non-contact sensors that can communicate information about emotional characteristics of users. For instance, the temperature of a human's body can convey information about their emotional state, and can be measured remotely (e.g., via an infrared temperature sensor). As another example, humidity can fluctuate as users sweat, and humidity can also be sensed remotely without revealing private information. Thus, features derived from temperature and/or humidity sensors can also be employed with the disclosed techniques.

In addition, other types of sensors can be employed, such photoplethysmography (PPG) sensors for heart rate, blood pressure, and/or blood oxygen level monitoring. Electroencephalogram (EEG) sensors can be employed to measure neurological activity, position tracking sensors can measure whether users' hands are shaky or their movements are jerky or smooth and relaxed, eye tracking sensors can detect eye movements and/or pupil dilation, etc. In some implementations, a wearable device is provided that incorporates a gas sensor as well as one or more other sensors, such as PPG or EEG sensors or eye tracking sensors.

While some of these additional sensor types may be more intrusive and/or implicate privacy concerns, there may be use cases where features derived from these sensors can improve prediction of emotional characteristics of users. For instance, users may be able to “opt-in” by explicitly agreeing to allow video, audio, and/or physiological sensor monitoring. In some cases, privacy can be preserved by including various sensors as components of a device (e.g., a virtual or augmented reality headset) that uses machine learning to predict emotional characteristics of a user locally, without ever transmitting that information to another device. Such a device could also delete any video, audio, or physiological sensor information immediately after use to preserve users' privacy.

As another consideration, note that the previous examples utilized training data having explicit user ratings of their own emotional characteristics. For adult humans without disabilities that impact their ability to communicate, this is an effective approach. However, consider a person with limited communication abilities, such as a coma patient or an infant. In some cases, implicit ratings can be obtained by other means. For instance, infant cries can be used as a proxy for negative emotions, e.g., gas and other sensor readings can be obtained over a period of time and a machine learning model could be trained using infant cries as an implicit label for negative emotions.

As another example, consider an animal such as livestock or a pet. This is another scenario where it could be difficult to obtain explicit ratings of emotional characteristics. However, dogs might yelp or whimper when distressed, or show distress postures such as a hunched back, tucked tail, and/or flattened ears. A computer vision model could be used to detect such postures and use them as an implicit label for negative emotions from a dog.

As another example, even users without communication difficulties may not want to be burdened by explicitly providing ratings of their own emotional characteristics. There are many different types of context that can be employed to infer the emotional characteristics of such users to derive implicit labels. For instance, if a user sends an email or text message, that email or text message can be evaluated using a natural language sentiment model for positive, neutral, or negative sentiment. If a user is sending very negative communications, this can indicate that the user is stressed or angry and thus an implicit label of negative emotions can be derived based on the detected sentiment. Conversely, if the user is sending happy or optimistic communications, an implicit label of positive emotions can be derived.

In addition, there are a wide range of actions that can be taken responsive to detecting the emotional characteristics of a user with a gas sensor. For instance, consider a computer that controls the HVAC, lighting, and/or sound equipment in a user's home or office. Some implementations can detect when a user is stressed and then take steps such as adjusting temperature or dispensing soothing scents (e.g., lavender) via the HVAC equipment, dimming the lights, and/or playing white noise or soothing music.

As another example, similar steps can be performed for a coma or surgery patient when distress is detected. In addition, medicine such as painkillers or sedatives can be automatically dispensed when a user is detected to be in distress. Alternatively, a clinician can be alerted to the patient's distress and the clinician can administer the medication.

As another example, consider a user driving a car. If the user is stressed, the user could be alerted, and a suggestion could be output for the user to take a break. As another example, a self-driving car might be driving in a manner that causes a user distress, e.g., the user may prefer to drive slower or have more following distance behind cars ahead of the self-driving car. In some cases, a self-driving car could adjust its own driving characteristics based on the emotional characteristics of the passengers.

As another example, consider a meeting scheduling application. By monitoring a user's emotional characteristics over time, it is possible to infer times when the user tends to be alert and/or happy versus distracted and/or annoyed. Thus, a scheduling application can take this into account and suggest scheduling meetings for the user at times when they tend to be alert or happy.

As another example, consider an electronic learning application where a child is given a periodic quiz on concepts they have learned. If the child is distressed during the quiz, the electronic learning application could automatically return to a tutorial portion and give the child a chance to slow down and internalize the lesson before trying the quiz again. In a similar vein, a generative language model could be utilized for automated storytelling of spooky Halloween stories, and the generative language model could make the story less scary if the audience (e.g., a young child) is showing signs of distress.

As another example, consider a market research application. As users view products on an online shopping website, their emotional characteristics could fluctuate depending on how they feel about the products being shown. Thus, in some cases, products returned in response to a user query could be re-ranked based on the user's emotional reaction. As another example, user profiles could be built by automatically inferring a user's preferences, etc.

In addition, note that user emotions also can serve as training data for other types of models. For instance, generative image models, generative language models, and/or generative multi-modal models can employ user emotions for model training purposes. One way to do so involves reinforcement learning from human feedback. Instead of or in addition to relying on human language or explicit ratings as a reinforcement learning signal for a generative model, some implementations can employ emotional characteristics of users detected via gas sensors as a reinforcement learning signal. Thus, a generative model can be updated based on emotional characteristics predicted from gas sensor readings as described elsewhere herein.

In some cases, individual emotional characteristics are mapped to specific actions using one or more rules. For example, in a regression approach, a rule could state if the user's predicted valence is below a numerical threshold (e.g., 3 or lower on a scale of 1-9) and their arousal is above a numerical threshold (e.g., 7 or higher on a scale of 1-9), then an action could be taken to help calm the user down. The selected action can be context-dependent. If the user is at home, the action could involve playing soothing music or dimming lights, if the user driving a car, the action could involve suggesting that the user take a short break, if the user is interacting with a generative language model, the action could involve prompting the generative language model to suggest relaxing activities or thoughts, etc. In a classification approach, individual emotions such as anger, happiness, or sadness could be mapped directly to corresponding actions without employing numerical thresholds. In other cases, a generative language model or multi-modal model can be prompted to suggest which actions should be taken based on the user's predicted emotional characteristics and other context information, such as their location, current activity (e.g., working, driving to or from work, texting a friend, etc.), currently-open applications, sentiment of their communications (e.g., drafting a friendly vs. rude email) etc.

In further implementations, the emotional characteristics of users can be tracked in relation to various stimuli to learn which stimuli tend to improve or harm the user's emotional state. For instance, if a user consistently exhibits high arousal and low valence when they email, text, or talk to a specific person, then this suggests that interacting with that person tends to hurt the emotional state of the user—e.g., make them angry. Thus, some implementations can suggest (e.g., via an on-screen message) that the user avoid communication with that person, particularly when the user is may already have somewhat elevated arousal and low valence. Conversely, if the user tends to have high valence and low arousal when interacting with another person, then another message can be provided to suggest that the user interact with that person instead, e.g., to help calm the user down. Similar approaches can be employed to suggest that a user avoid video or audio content that tends to cause negative emotional reactions, or to suggest that the user consume other video or audio content that tends to calm the user when they already appear stressed. Said another way, some implementations can selectively prompt the user to avoid interactions with people or electronic content that have previously caused the user to have negative emotional reactions, and to suggest that the user partake in interactions with people or electronic content that have previously caused the user to have positive emotional reactions.

Furthermore, note that there can be some variation in gas compositions of different users. Thus, in some implementations, a machine learning model is first pretrained using training data obtained from a group of users. Then, the pretrained model can then be tuned to other users on an individual basis. For instance, a user can perform an enrollment process where they consume emotional content and provide emotional characteristic ratings while gas levels are monitored. Then, the pretrained model can be tuned for that user using the gas levels and emotional characteristic ratings from the enrollment session as additional training data.

### Technical Effect

As described above, the disclosed implementations offer significant improvements in user interaction with a computing device. By inferring the emotional characteristics of a user via gas sensor readings, it is possible to cause a computer to take a wide range of actions that are appropriate given the predicted emotional characteristics of the user. As a consequence, the user's interaction with the computer can be improved. Users can be offered more targeted computer experiences, external systems such as vehicles, HVAC, and lighting can be controlled, etc.

In addition, as noted previously, some sensors can be intrusive. For instance, EEG sensors or PPG sensors generally contact a user's skin, which is disfavored by some users. In contrast, gas sensing can be performed without any contact with the user's body, which is preferred by many users. Furthermore, some biological signals such as blood pressure might be considered private information by many users. However, most users do not consider gas levels in their breath or environment to be particularly sensitive information. Likewise, gas sensing has very significant privacy benefits in contrast to technologies that involve video or audio monitoring of a user's environment.

### Device Implementations

As noted above with respect to FIG. 1, system 100 includes several devices, including a wearable device 110, a client device 120, a server 130, and a server 140. As also noted, not all device implementations can be illustrated, and other device implementations should be apparent to the skilled artisan from the description above and below.

The term “device”, “computer,” “computing device,” “client device,” and or “server device” as used herein can mean any type of device that has some amount of hardware processing capability and/or hardware storage/memory capability. Processing capability can be provided by one or more hardware processors (e.g., hardware processing units/cores) that can execute computer-readable instructions to provide functionality. Computer-readable instructions and/or data can be stored on storage, such as storage/memory and or the datastore. The term “system” as used herein can refer to a single device, multiple devices, etc.

Storage resources can be internal or external to the respective devices with which they are associated. The storage resources can include any one or more volatile or non-volatile memory, hard drives, flash storage devices, and/or optical storage devices (e.g., CDs, DVDs, etc.), among others. As used herein, the term “computer-readable medium” can include signals. In contrast, the term “computer-readable storage medium” excludes signals. Computer-readable storage media includes “computer-readable storage devices.” Examples of computer-readable storage devices include volatile storage media, such as RAM, and non-volatile storage media, such as hard drives, optical discs, and flash memory, among others.

In some cases, the devices are configured with a general-purpose hardware processor and storage resources. In other cases, a device can include a system on a chip (SOC) type design. In SOC design implementations, functionality provided by the device can be integrated on a single SOC or multiple coupled SOCs. One or more associated processors can be configured to coordinate with shared resources, such as memory, storage, etc., and/or one or more dedicated resources, such as hardware blocks configured to perform certain specific functionality. Thus, the term “processor,” “hardware processor” or “hardware processing unit” as used herein can also refer to central processing units (CPUs), graphical processing units (GPUs), neural processing units (NPUs), controllers, microcontrollers, processor cores, or other types of processing devices suitable for implementation both in conventional computing architectures as well as SOC designs.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

In some configurations, any of the modules/code discussed herein can be implemented in software, hardware, and/or firmware. In any case, the modules/code can be provided during manufacture of the device or by an intermediary that prepares the device for sale to the end user. In other instances, the end user may install these modules/code later, such as by downloading executable code and installing the executable code on the corresponding device.

Also note that devices generally can have input and/or output functionality. For example, computing devices can have various input mechanisms such as keyboards, mice, touchpads, voice recognition, gesture recognition (e.g., using depth cameras such as stereoscopic or time-of-flight camera systems, infrared camera systems, RGB camera systems or using accelerometers/gyroscopes, facial recognition, etc.). Devices can also have various output mechanisms such as printers, monitors, etc.

Also note that the devices described herein can function in a stand-alone or cooperative manner to implement the described techniques. For example, the methods and functionality described herein can be performed on a single computing device and/or distributed across multiple computing devices that communicate over network(s) 150. Without limitation, network(s) 150 can include one or more local area networks (LANs), wide area networks (WANs), the Internet, and the like.

### ADDITIONAL EXAMPLES

Various device examples are described above. Additional examples are described below. One example includes a method comprising obtaining gas sensor readings from a gas sensor, the gas sensor being located in a vicinity of a user when the gas sensor readings are obtained, obtaining gas level features, the gas level features being based on the gas sensor readings, inputting the gas level features to a trained machine learning model, receiving, from the trained machine learning model, a predicted emotional characteristic of the user, and causing a computer to perform an action based at least on the predicted emotional characteristic of the user.

Another example can include any of the above and/or below examples where the gas sensor readings include first gas sensor readings obtained from a wearable device that incorporates a first gas sensor.

Another example can include any of the above and/or below examples where the gas sensor readings include second gas sensor readings obtained from a second gas sensor measuring ambient gas levels in a room with the user.

Another example can include any of the above and/or below examples where the trained machine learning model comprises a random forest or a neural network.

Another example can include any of the above and/or below examples where the gas level features identify at least one of nitrogen dioxide levels, ethyl alcohol levels, volatile organic compound levels, or carbon monoxide levels.

Another example can include any of the above and/or below examples where the gas level features identify changes over time to at least one of the nitrogen dioxide levels, the ethyl alcohol levels, the volatile organic compound levels, or the carbon monoxide levels.

Another example can include any of the above and/or below examples where the method further comprises obtaining respiration features from the gas sensor readings, the respiration features relating to duration or intensity of respiration by the user and inputting the respiration features into the trained machine learning model with the gas level features.

Another example can include any of the above and/or below examples where the method further comprises obtaining context features relating to a context of the user and inputting the context features to the trained machine learning model with the gas level features.

Another example can include any of the above and/or below examples where the method further comprises obtaining bio-signal features relating to a physiological state of the user and inputting the bio-signal features to the trained machine learning model with the gas level features.

Another example can include any of the above and/or below examples where the causing comprises communicating the predicted emotional characteristic from an operating system to an application via an application programming interface, where the application, responsive to receiving the predicted emotional characteristic from the operating system via the application programming interface, performs at least one of: adjusting behavior of at least one automated agent assisting the user, triggering an alert regarding the user, or controlling an environment where the user is located.

Another example includes a method comprising obtaining training data, the training data including gas sensor readings from a gas sensor, the gas sensor being located in a vicinity of a user when the gas sensor readings are obtained and emotional characteristic ratings indicating emotional characteristics of the user when the gas sensor readings are obtained, obtaining gas level features, the gas level features being based on the gas sensor readings, training a machine learning model to perform gas sensor-based prediction of emotional characteristics based on the gas level features and the emotional characteristic ratings, and outputting the trained machine learning model.

Another example can include any of the above and/or below examples where the method further comprises presenting emotional content to the user while obtaining the gas sensor readings.

Another example can include any of the above and/or below examples where the emotional content includes videos.

Another example can include any of the above and/or below examples where the method further comprises tuning the trained machine learning model to another user based on other training data for the another user, the other training data including other gas sensor readings and other emotional characteristic ratings obtained for the another user.

Another example can include any of the above and/or below examples where the method further comprises obtaining one or more of respiration features, context features, or bio-signal features relating to the user and training the machine learning model based on the gas level features and the one or more of the respiration features, the context features, or the bio-signal features.

Another example includes a system comprising a processor and a computer-readable storage medium storing instructions which, when executed by the processor, cause the system to obtain gas sensor readings from a gas sensor, the gas sensor being located in a vicinity of a user when the gas sensor readings are obtained, obtain gas level features based on the gas sensor readings, input the gas level features to a trained machine learning model, receive, from the trained machine learning model, a predicted emotional characteristic of the user, and perform an action based at least on the predicted emotional characteristic of the user.

Another example can include any of the above and/or below examples where the action involves displaying content to the user.

Another example can include any of the above and/or below examples where the action involves outputting an alert regarding the predicted emotional characteristic.

Another example can include any of the above and/or below examples where the trained machine learning model comprises a deep neural network or a random forest.

Another example can include any of the above and/or below examples where the system comprises a wearable device that includes the gas sensor.

### CONCLUSION

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims and other features and acts that would be recognized by one skilled in the art are intended to be within the scope of the claims.

1. A method comprising:
obtaining gas sensor readings from a gas sensor, the gas sensor being located in a vicinity of a user when the gas sensor readings are obtained;
obtaining gas level features, the gas level features being based on the gas sensor readings;
inputting the gas level features to a trained machine learning model;
receiving, from the trained machine learning model, a predicted emotional characteristic of the user; and
causing a computer to perform an action based at least on the predicted emotional characteristic of the user.

2. The method of claim 1, the gas sensor readings including first gas sensor readings obtained from a wearable device that incorporates a first gas sensor.

3. The method of claim 2, the gas sensor readings including second gas sensor readings obtained from a second gas sensor measuring ambient gas levels in a room with the user.

4. The method of claim 1, the trained machine learning model comprising a random forest or a neural network.

5. The method of claim 1, the gas level features identifying at least one of nitrogen dioxide levels, ethyl alcohol levels, volatile organic compound levels, or carbon monoxide levels.

6. The method of claim 5, the gas level features identifying changes over time to at least one of the nitrogen dioxide levels, the ethyl alcohol levels, the volatile organic compound levels, or the carbon monoxide levels.

7. The method of claim 1, further comprising:
obtaining respiration features from the gas sensor readings, the respiration features relating to duration or intensity of respiration by the user; and
inputting the respiration features into the trained machine learning model with the gas level features.

8. The method of claim 1, further comprising:
obtaining context features relating to a context of the user; and
inputting the context features to the trained machine learning model with the gas level features.

9. The method of claim 1, further comprising:
obtaining bio-signal features relating to a physiological state of the user; and
inputting the bio-signal features to the trained machine learning model with the gas level features.

10. The method of claim 1, wherein the causing comprises:
communicating the predicted emotional characteristic from an operating system to an application via an application programming interface,
wherein the application, responsive to receiving the predicted emotional characteristic from the operating system via the application programming interface, performs at least one of:
adjusting behavior of at least one automated agent assisting the user,
triggering an alert regarding the user, or
controlling an environment where the user is located.

11. A method comprising:
obtaining training data, the training data including:
gas sensor readings from a gas sensor, the gas sensor being located in a vicinity of a user when the gas sensor readings are obtained; and
emotional characteristic ratings indicating emotional characteristics of the user when the gas sensor readings are obtained;
obtaining gas level features, the gas level features being based on the gas sensor readings;
training a machine learning model to perform gas sensor-based prediction of emotional characteristics based on the gas level features and the emotional characteristic ratings; and
outputting the trained machine learning model.

12. The method of claim 11, further comprising:
presenting emotional content to the user while obtaining the gas sensor readings.

13. The method of claim 12, the emotional content including videos.

14. The method of claim 11, further comprising:
tuning the trained machine learning model to another user based on other training data for the another user, the other training data including other gas sensor readings and other emotional characteristic ratings obtained for the another user.

15. The method of claim 11, further comprising:
obtaining one or more of respiration features, context features, or bio-signal features relating to the user; and
training the machine learning model based on the gas level features and the one or more of the respiration features, the context features, or the bio-signal features.

16. A system comprising:
a processor; and
a computer-readable storage medium storing instructions which, when executed by the processor, cause the system to:
obtain gas sensor readings from a gas sensor, the gas sensor being located in a vicinity of a user when the gas sensor readings are obtained;
obtain gas level features based on the gas sensor readings;
input the gas level features to a trained machine learning model;
receive, from the trained machine learning model, a predicted emotional characteristic of the user; and
perform an action based at least on the predicted emotional characteristic of the user.

17. The system of claim 16, wherein the action involves displaying content to the user.

18. The system of claim 16, wherein the action involves outputting an alert regarding the predicted emotional characteristic.

19. The system of claim 16, the trained machine learning model comprising a deep neural network or a random forest.

20. The system of claim 16, the system comprising a wearable device that includes the gas sensor.
